(19) United States
(12) Patent Application Publication
(10) Pub. No.: US 2022/0355237 A1
(43) Pub. Date: Nov. 10, 2022

(54) CAPTURING ATMOSPHERIC GAS WITH A DISTRIBUTED SYSTEM

(71) Applicant: Kevin L. Thomas Capital LLC, Lauderdale Lakes, FL (US)

(72) Inventor: Kevin L. Thomas, Lauderdale Lakes, FL (US)

(73) Assignee: Kevin L. Thomas Capital LLC, Lauderdale Lakes, FL (US)

(21) Appl. No.: 17/720,650

(22) Filed: Apr. 14, 2022

Related U.S. Application Data
(60) Provisional application No. 63/184,244, filed on May 5, 2021.

(51) Int. Cl. B01D 53/04; G01N 33/00

(52) U.S. Cl. CPC B01D 53/0454; G01N 33/0075; B01D 53/0407; B01D 53/0462; B01D 2257/504; B01D 2259/40009; B01D 2259/40092

(57) ABSTRACT

Deleterious gas is captured from atmospheric air using capture units dispersed across a geographic region. Each unit has a filter that is capable of capturing compounds from the gas from air when air is passed through the filter by fans. The units additionally include a sensor for sensing a level of the gas in the air. An electronic processor controls the fan, and communicates data from the sensor to other units and/or a central electronic processor. The electronic processors of the units or the central processor controls the fan speed of units in areas of higher concentration of the gas, selecting which units to become active based upon a proximity of each unit to the gas concentration, as well as a direction of movement of the concentration. A communicated presence of errors or a low battery state of a unit, is used by the processor to select other units nearby for operation instead of the affected unit.

### FIELD OF THE DISCLOSURE

This disclosure relates to capturing atmospheric gas, and more particularly, to a network of devices which cooperate for capturing carbon dioxide or other deleterious gases.

### BACKGROUND OF THE DISCLOSURE

Atmospheric carbon dioxide (CO2) levels contribute to a rise in global temperatures by reflecting sunlight that is reflected from the Earth's surface back towards the earth. Carbon dioxide levels in the atmosphere have been increasing in recent years at a rate which causes environmental change at a rate that is too rapid to allow for gradual changes in land use and lifestyle. As a result, destructive natural forces and agricultural changes have created unusual widescale human suffering.

Devices are known which are targeted to capturing carbon at point sources, or sites which generate large amounts of CO2, such as power plants or factories. One such system uses excess heat generated at the site to provide energy to power aspects of the system.

### SUMMARY OF THE DISCLOSURE

In an embodiment of the disclosure, a method for capturing a deleterious gas from atmospheric air comprises positioning a plurality of deleterious gas capturing units within a region producing the deleterious gas. Each unit includes a filter of a type capable of capturing the deleterious gas from air when air is passed through the filter, one or more fans for passing air from the atmosphere through the filter, the unit being in an active status when air is passed through the filter, and an inactive status when air is not being passed through the filter, a sensor for sensing a level of the deleterious gas in the air, a processor circuit connected to the sensor and the fans to obtain data from the sensor and control operation of the fan, and a communication circuit responsive to the processor circuit to communicate data between the unit and at least one other unit; and programming the processor of each unit to cause the communication circuit to communicate with a plurality of other units to communicate data including a concentration of deleterious gas in the air as measured by the sensor of the unit and whether the unit is in an active status; programming the processor of each unit to independently determine an amount of air to pass through the filter based upon (a) a concentration of deleterious gas sensed by the sensor of the unit, and (b) a concentration of deleterious gas sensed by, as well as an activity status of, a plurality of other units in communication with the unit; the independent determination based upon a calculation of maximizing yield of the unit and the plurality of units with which the unit is communicating.

In variations thereof, wherein communicating with a plurality of other units includes communicating information pertaining to yield of the deleterious gas, and determining is further based upon yield of other units; whereby the one or more fans are configured to have an adjustable rate of operation, and whereby independently determining an amount of air to pass through the filter includes determining a rate of operation of the one or more fans; whereby determining a rate of operation of the one or more fans includes determining a number of fans which are operating; and/or whereby determining a rate of operation of the one or more fans includes determining a speed at which one or more fans are operating.

In further variations thereof, each unit further includes a purge storage container and a source of hot water connected to the filter, hot water admissible into the filter under the control of the processor to purge the filter of components of the deleterious gas that has been captured and to pass the components gas into the purge storage container.

In other variations thereof, wherein the activity status includes whether one or more units are not functioning; wherein the activity status includes whether one or more units are functioning at a maximal rate; and/or wherein the deleterious gas includes a carbon compound.

In another embodiment of the disclosure, a method for capturing a deleterious gas from atmospheric air, comprises positioning a plurality of deleterious gas capturing units within a region producing the deleterious gas, each unit including a filter of a type capable of capturing compounds from the deleterious gas from air when air is passed through the filter, one or more fans for passing air from the atmosphere through the filter, the unit being in an active status when air is passed through the filter, and an inactive status when air is not being passed through the filter, a sensor for sensing a level of the deleterious gas in the air, and a communication circuit; and programming one or more electronic processors to a) communicate with each of a plurality of units to collect data from the sensor of each of the plurality of units using the communication circuit of each unit; b) use the collected data to determine if the deleterious gas is present at a level above a predetermined level at each of the units; c) determine at least one set of units of a predetermined set size, each of the units of the set having the predetermined level of deleterious gas present; and d) communicate to each of the set of units to operate one or more fans of the unit to capture deleterious gas using the filter of the unit; and e) periodically repeat steps (a)-(d) whereby units are operated only where and while the predetermined level of deleterious gas is present at a plurality of units.

In variations thereof, each unit further includes a battery configured to operate the electronic processors, the one or more fans and the sensor; the one or more electronic processors are further programmed to communicate with each of the plurality of units to determine an amount of battery capacity of the units, and using the determined battery capacity to determine members of the set of units of a predetermined size, the members of the set including only units having a predetermined amount of battery capacity; and/or wherein in step (d), the one more electronic processors communicate to each of the set of units a number of the one or more fans to be operated, or a rate of speed of the one or more fans to be operated, to achieve a total fan output corresponding to the predetermined level of deleterious gas present. In further variations thereof, each unit further includes a battery configured to operate the one or more fans and sensor, the number of fans to be operated or the rate of speed of the one or more fans corresponding to a charge level of the battery; wherein if a first unit in the set of units has a charge level below a predetermined charge level, and a charge level of a unit not in the set of units that is near to the first unit is above a predetermined charge level, the one or more processors includes the unit that is near in the set; each unit further including a means of removing captured gas compounds from the filter; and/or each unit further including a means of recording errors in the operation of the unit.

In other variations thereof, the method further includes using the communication circuit to communicate the recorded errors to the one or more electronic processors; the one or more electronic processors changing members of the set in response to recorded errors communicated;

and/or wherein the one or more electronic processors changes the set of units based upon a predicted direction of movement of the predetermined level of deleterious gas away from the set based upon communication of deleterious gas levels communicated from units outside of the set.

### BRIEF DESCRIPTION OF THE DRAWINGS

A more complete understanding of the disclosure, and the attendant advantages and features thereof, will be more readily understood by reference to the following detailed description when considered in conjunction with the accompanying drawings, in which:

FIG. 1 depicts a front view of a gas capturing unit of the disclosure;

FIG. 2 depicts a rear view of the unit of FIG. 1;

FIG. 3 depicts a top view of the unit of FIG. 1;

FIG. 4 depicts a back view of the unit of FIG. 1;

FIG. 5 depicts a top view of the unit of FIG. 1, with a top cover removed, and showing only the battery, filters, and purge collection container;

FIG. 6 depicts a flow diagram for optimizing gas capture by a unit as depicted in FIG. 1;

FIGS. 7-8 depict a self-diagnosis function of a unit as depicted in FIG. 1; and

FIGS. 9-10 depict a plurality of units in a geographic area, with operating units depicted as hollow circles, non-operating units depicted as solid circles, and a wavy encircling region as an area of relatively higher unwanted gas concentration in an area, with FIG. 10 showing a change in operation of units based upon a shift of the gas concentration in the area, the change in operation of the units based upon swarm intelligence.

### DETAILED DESCRIPTION OF THE DISCLOSURE

This written description uses examples to disclose the embodiments, including the best mode, and also to enable those of ordinary skill in the art to make and use the invention. The patentable scope is defined by the claims, and can include other examples that occur to those skilled in the art. Such other examples are intended to be within the scope of the claims if they have structural elements that do not differ from the literal language of the claims, or if they include equivalent structural elements with insubstantial differences from the literal languages of the claims.

Note that not all of the activities described above in the general description or the examples are required, that a portion of a specific activity may not be required, and that one or more further activities can be performed in addition to those described. Still further, the order in which activities are listed are not necessarily the order in which they are performed.

In the foregoing specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

It can be advantageous to set forth definitions of certain words and phrases used throughout this patent document. The term “communicate,” as well as derivatives thereof, encompasses both direct and indirect communication. The term “discreet,” as well as derivatives thereof, references to the amount of skin exposed by a user of the garment, rather than the type of style of the garment. The terms “include” and “comprise,” as well as derivatives thereof, mean inclusion without limitation. The term “or” is inclusive, meaning and/or. The phrase “associated with,” as well as derivatives thereof, can mean to include, be included within, interconnect with, contain, be contained within, connect to or with, couple to or with, be communicable with, cooperate with, interleave, juxtapose, be proximate to, be bound to or with, have, have a property of, have a relationship to or with, or the like. The phrase “at least one of,” when used with a list of items, means that different combinations of one or more of the listed items can be used, and only one item in the list can be needed. For example, “at least one of: A, B, and C” includes any of the following combinations: A, B, C, A and B, A and C, B and C, and A and B and C. Also, the use of “a” or “an” are employed to describe elements and components described herein. This is done merely for convenience and to give a general sense of the scope of the invention. This description should be read to include one or at least one and the singular also includes the plural unless it is obvious that it is meant otherwise.

The description in the present application should not be read as implying that any particular element, step, or function is an essential or critical element that must be included in the claim scope. The scope of patented subject matter is defined only by the allowed claims. Moreover, none of the claims invokes 35 U.S.C. § 112(f) with respect to any of the appended claims or claim elements unless the exact words “means for” or “step for” are explicitly used in the particular claim, followed by a participle phrase identifying a function.

Benefits, other advantages, and solutions to problems have been described above with regard to specific embodiments. However, the benefits, advantages, solutions to problems, and any feature(s) that can cause any benefit, advantage, or solution to occur or become more pronounced are not to be construed as a critical, required, sacrosanct or an essential feature of any or all the claims.

After reading the specification, skilled artisans will appreciate that certain features are, for clarity, described herein in the context of separate embodiments, can also be provided in combination in a single embodiment. Conversely, various features that are, for brevity, described in the context of a single embodiment, can also be provided separately or in any subcombination. Further, references to values stated in ranges include each and every value within that range.

As used herein, the term “about” or “approximately” applies to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. As used herein, the terms “substantial” and “substantially” means, when comparing various parts to one another, that the parts being compared are equal to or are so close enough in dimension that one skill in the art would consider the same. Substantial and substantially, as used herein, are not limited to a single dimension and specifically include a range of values for those parts being compared. The range of values, both above and below (e.g., “+/−” or greater/lesser or larger/smaller), includes a variance that one skilled in the art would know to be a reasonable tolerance for the parts mentioned.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

Headings are provided for the convenience of the reader, and are not intended to be limiting in any way.

The inventor has found that because existing systems for removing carbon from the atmosphere are very large and expensive, they are limited in number, and are thus positioned at few locations. However, excess atmospheric gases diffuse into the atmosphere, and may become concentrated over a wide area that is not close to a point source which is emitting deleterious gas into the atmosphere. As a result, deleterious atmospheric gases are not captured efficiently. Accordingly, the disclosure provides a network of systems which are relatively much smaller and much less expensive, and which can be distributed over a wide area, and which can operate as needed where the deleterious atmospheric gases have concentrated.

Systems of the disclosure capture deleterious or excess gas from the atmosphere by drawing in air and passing the air through a filter which binds to the excess gas, thereby discharging air which has a lower concentration of the excess gas. The disclosure additionally provides system logic which enables an overall amount of air intake across a number of systems to scale with excess gas concentrations, thus allowing optimization of energy use, maximization of excess gas capture, and cost reduction. Systems of the disclosure are able to operate at a negative net excess gas result, whereby an amount of excess gas released into the atmosphere as a result of producing energy for the systems is less than the amount of excess gas capture.

Where the excess gas is CO2 or other carbon containing gas, a net negative carbon result is achieved. Examples of other deleterious gases that can be reduced in accordance with the disclosure include, but are in no means limited to, CO, Propane, Methane, Hydrogen Fluoride, Hydrogen Sulfide, Volatile Organic Compounds (VOCs), and other harmful, undesirable, explosive, or otherwise harmful gases.

Throughout this disclosure, reducing an excess atmospheric gas of carbon is described; however it should be understood that the disclosure can be used for other gases, wherein the method of filtration and filter cleaning may differ, but the remaining devices and methods are as described herein.

Atmospheric CO2 levels are understood to contribute to the rise in global temperatures. The inventor has determined that previously known systems and processes have a net negative effect on reducing carbon, as the amount of energy required to build and operate such systems cause the production of more atmospheric carbon than is recaptured. The disclosure provides a system which captures far more carbon that is created in building and operating the system.

In an embodiment of the disclosure, a system of relatively small, discreet systems are mutually connected by a communication network, and use sophisticated or artificial intelligence software to collectively maximize the efficient capture of a target deleterious gas, such as carbon. In particular, in densely populated areas, the system will mitigate and reduce the concentration of CO2 and/or other deleterious gas closer to the source, such as motor vehicles, and before it has time to defuse into the atmosphere.

In an embodiment, this intelligent networked design allows for capacity loading and intake adjustment based on the local need, limiting excess energy consumption and maximizing filtration where it is needed most. As detailed further herein, capacity loading relates to the number of units that are operating to remove carbon, and intake adjustment relates to the rate at which individual units are operating.

In accordance with the disclosure, many gas capture units 100 are spread over a large area, for example within a community with many internal combustion based motor vehicles. These units 100 are at least small enough to be supported by typical building roofs without reinforcement, for example the size of a typical window air conditioner, which is typically under 100 pounds. This differs from previously known systems which are large, for example a maximum transportable size, e.g. one or more modules that are tractor trailer load sized, or much larger structures which are assembled or built on-site. These larger structures are typically standalone units, and would typically be located near a factory which produces carbon gas as waste.

As a result, the disclosure provides a solution for capturing carbon that is emitted from many sources over a wide area, on a scale of miles, each unit being much smaller and more cost-effective as a direct air capture endpoint, the units collectively being networked on a large scale, as is a distribution grid for a public utility. It would be impractical and prohibitively expensive to place many of the large plant-like solutions throughout a city, and as such, they are unsuitable for implementation close to a widely distributed source of CO2 production.

Individual units are interconnected by a computer network, and in an embodiment leverage swarm intelligence (SI) to optimize the energy consumption to CO2 collection ratio. SI is the collective behavior of decentralized, self-organized systems, which may be natural or artificial. The concept is employed in work on artificial intelligence, in which SI systems consist typically of a population of simple agents or “boids”, in this case represented by individual units 100, interacting and impacted locally based on activity of their nearest neighbor units 100, and based on their local environment and operational status. By leveraging SI a system 10 of a plurality of units 100 utilizes smaller more energy efficient devices that communicate with surrounding units to impact local concentrations of deleterious gas using the least amount of energy. When elevated CO2 levels are detected by a unit 100, this measurement is validated by detected levels as measured by neighboring units 100, whereby system 10 engages as a collective swarm to target dynamic CO2 concentrations for optimal yield (captured gas) and efficiency. Likewise, when the collective detects reduced CO2 levels the system will reduce its energy consumption by reducing the number of units 100 which are capturing, and/or reduce the rate at which individual units 100 are capturing.

Carbon dioxide production, for example, is not evenly distributed globally. Large carbon capture systems located only at major point sources are not preventing the release into the upper atmosphere of carbon gas released at other locations where carbon is currently being released in high overall quantities. By providing units 100 of reduced size, and by providing a system logic that allows for intake to scale with CO2 or deleterious gas concentrations, energy use is optimized, CO2 or deleterious gas capture is maximized, and cost of operations is reduced for a negative net carbon result.

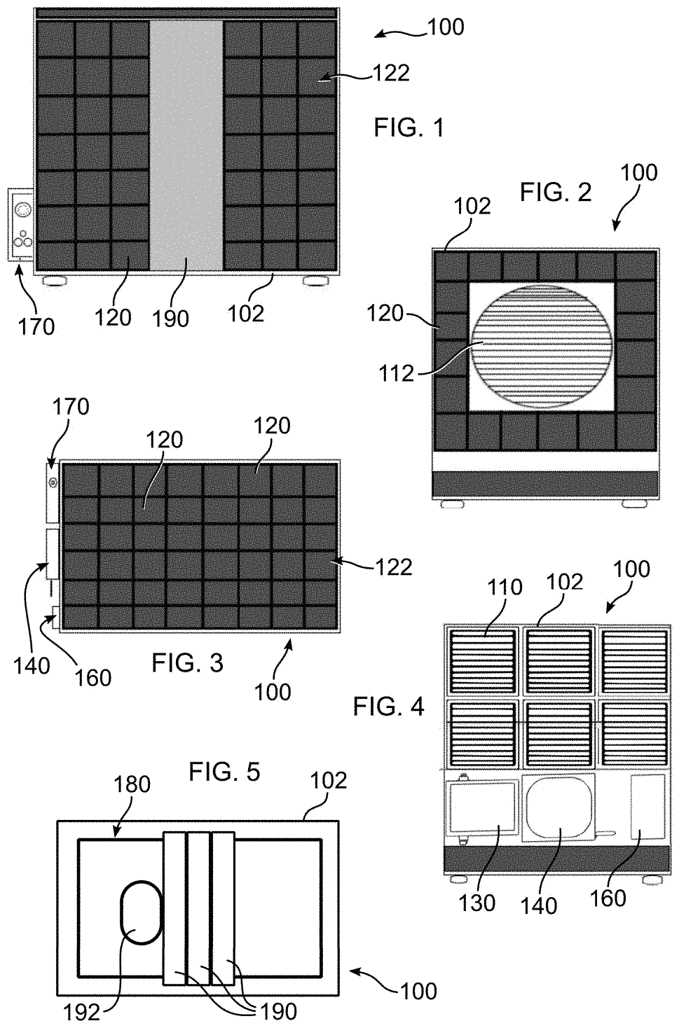

With reference to FIGS. 1-5, a unit 100 can include at least one of each of an intake fan 110, photovoltaic/solar cell 120, electric on-demand water heater 130, wireless communication circuit 140, processor circuit 160, sensors 170, including a gas sensor for the gas to be captured, energy storage/battery 180, and gas filter 190.

Where convenient, utility power or other source of electrical power can be substituted for the solar cell 120 and battery 180, although these elements greatly increase ease of deployment and thus acceptance, and ensure that systems are not accidentally or intentionally deprived of power. For example, a provider of electrical energy may believe it is saving energy by turning off power to a unit 100 periodically; however this can have an adverse effect on the efficiency of the system 10 to which the unit 100 belongs. If units 100 do not rely on a contribution other than some unused space, concerns about cost or other obligation are reduced.

In an embodiment, intake fan 110 is a DC powered fan, which is combined in an array of fans, for example 6 fans, although as little as two fans or many more than six fans can be used, depending upon the amount of air it is desired to process for a given amount of time. Intake fans 110 can also be variable speed fans, controlled by processor circuit 160, which further impacts the number of fans needed. Where variable speed fans are used, it is helpful to efficiency if fans 110 can operate efficiently at all speeds. If fans 110 are most efficient as single speed fans, the overall air movement/air intake can be controlled by powering additional fans as a requirement for air intake increases.

In one embodiment, photovoltaic cells 120 are aligned in an array, for example provided as a solar panel 122, mounted to an outside surface of the unit 100, for example a sky facing surface, to thereby absorb sunlight to generate power for unit 100, to enable a self-contained and closed power system. Electrical power generated by cells 120 in excess of what is currently required by unit 100 can be stored in battery 180 for future use, and particularly for use during non-daylight hours, or for intervals of power requirement which exceed a maximum solar output. Battery 180 can be provided in an easily replaceable configuration so that batteries 180 can be exchanged when they approach a maximum number of charge cycles, or when they otherwise do not store sufficient power for efficient operation of unit 100.

Battery 180 can include a single battery of appropriate voltage and power capacity, or can be a plurality of batteries connected in series or parallel to thereby provide the desired voltage and capacity. Where a plurality of batteries are used, they can be combined into a single package to facilitate exchange. A charge controller connected to the solar cell(s) 120 or other source of electricity can manage the charging voltage and rate to perform active balancing which can optimize battery life and performance of battery 180.

In an embodiment, hot water is used to collect captured carbon from gas filter 190. Accordingly, electrical on-demand water heater 130 generates hot water, which is passed through filter 190 to purge captured carbon trapped in filter 190. In an embodiment, a supply of cold water is connected to unit 100, or unit 100 can contain a supply of water in a tank (not shown). The water supply is passed into heater 130 where it passes over a heating element to reach the target temperature before being released by heater 130 to produces the necessary quantity of hot water as needed, which reduces the size of the water heater components. Alternatively, a tank can be provided to hold a quantity of heated water.

The heated water is passed through filter 190 to transfer or purge the CO2 into the water. After leaving the filter, the water/effluent from the purge can be collected and stored for periodic collection. Where gas is release from the effluent, this gas can be compressed using a pump and stored in a tank. Alternatively, collected effluent can be stripped of carbon in centralized facilities, and the carbon or other gas can be fixed in solid form for reuse or can be stored, or sold as gas for consumption/recycling. Based on sensors 170 associated with purge storage container 192, processor circuit 160 can notify a server as described herein when the storage of effluent is full and needs to be collected.

The temperature of the heated water is selected to produce the optimum result during filtration, for example about 100 C for filtration of CO2. As required, heater 130 can produce superheated water, or water at substantially less than the boiling point. Other liquids can be heated using heater 130, and these liquids may be heated to a substantially high temperature than 100 C.

Wireless communication circuit 140 can transmit and receive electromagnetic energy, to communicate at frequencies corresponding to a communication protocol selected. Examples of architecture or protocol supported can include one or more of cellular such as LTE, GSM, 4G or 5G, WLAN, WiFi, Zigbee, Modbus, Bluetooth, Sigfox, and PAN. It should be understood that new communications protocols and methods are evolving, and that this list is merely representative of some of the currently available technologies that can be used as part of the disclosure.

These protocols can be categorized as operating at three levels—within unit 100, between units 100, and between one or more units 100 and the Internet or other wide area network and to related servers. Each unit 100 can be represented as an IoT device (Internet of Things) and can use corresponding protocols to communicate locally to neighboring units, or to communicate directly to the Internet. A long range communication network, such as cellular or an ISP or other WAN, can provide a connection to the Internet, and a lower power network, such as Sigfox, can be used to communicate to neighboring units 100. In this manner, only certain ones of unit 100 need to have longer distance communication capabilities, or even an ability to communicate on the Internet. Neighboring units can relay data to other units 100, until data arrives at an Internet connected unit 100, reducing overall cost of system 10. This communication can be random, or organized. For example, where a nearest neighbor unit 100 is known, communication can be established only with a single unit, provided a chain is formed between all units 100 relying on a given unit 100 responsible for forwarding data to the Internet. While wireless communication facilitates and simplifies installation and acceptance, it should be understood that units 100 can be mutually connected or connected to the Internet using a wired connection where convenient and reliable.

Units 100 communicate with each other, in accordance with the disclosure, to optimize coordination to address local high concentrations of deleterious atmospheric gases as they arise. Thus, a wireless protocol is selected which uses the least amount of energy while providing adequate range and bandwidth for the data to be communicated.

In accordance with the disclosure, wireless communication circuit 140 can include more than one type of wireless transmitter/receiver, each associated with a processor that is capable of maintaining the relevant protocol. Each type can be provided as a replaceable module, so that the abilities of a given unit 100 can be selected for the role it is intended to play in a system 100. For example, a unit 100 may only be intended to communicate with one or more of its nearest neighbor units, or only neighboring units within range, while other units can be configured to communicate over a long distance network such as a cellular network connected to the Internet. The long distance connected units can communicate over the wide area network or Internet to (a) join with/share data with another cluster of units, (b) join with another cellular connected unit that is part of the same swarm, thereby (i) uniting the entire swarm or (ii) improving speed of communication within the swarm, (c) to report data to a server, or (d) to enable swarm behavior to be modified by the server.

Data reported to the server can include, at least, (a) maintenance status or requirements for one or more units, (b) performance data for one or more units, (c) performance for one or more swarms of units, (d) a request to collect effluent or captured gas from one or more units, (e) measured gas concentrations at the location of one or more units, (f) other sensed conditions at the location of one or more units. Servers can send data to connected units to, at least, (1) request specific data from one or more units (1) relay any of the foregoing data in (a-f) to one or more units, (2) command certain actions of one or more units, and (3) reprogram one or more units. When it is needed for a server to communicate to a unit which is not connected to the server via a LAN or WAN or other method, the server can relay such a request to a unit that is connected to the server, and that unit can relay the request to the target unit using any of the methods by which the swarm communicates.

Units 100 include a processor circuit 160, which should be understood to include one or more electronic processors, which functions at least to manage wireless communication, and to act upon sensor data to control operation of unit 100 via various actuators, as is known in the art. However, processor circuit 160 can carry out other functions, such as gather data from other components of unit 100 for reporting, control the functioning of other components of unit 100, and perform data calculations helpful to the efficient operation of one or more units. These calculations can alternatively be carried out by a connected server, or be carried out by one or more units 100, or be carried out by one or more connected servers. Advantageously processor circuit 160 can be conveniently interchanged or upgraded, as well the for the other components, so that units 100 can perform as efficiently as possible over an extended period of time, benefiting from advancements in technology. Processor circuit 160 and communication circuit 140 can be combined into a single module, board or assembly, or can be provided as a plurality of such.

In one embodiment, sensors 170 includes one or more CO2 sensors which are used to measure the atmospheric levels of CO2 in the vicinity of a unit 100 with which the CO2 sensor is connected. This data can be used to control operation of the connected unit 100, as well as other units 100, via a local network or through a wide area network and via a server, as described elsewhere herein. Sensors 170 can further include a temperature sensor, which can provide data used to control the operation of unit 100. Additional sensors 170 can include temperature, position, and other sensors specifically associated with other components within unit 100, and such sensors can provide data to processor circuit 160.

In an embodiment, in order to determine the necessary intake rate, sensors 170 measure the saturation of CO2 in the surrounding air, which data can be provided in parts per million (PPM) or other scale. This measurement is passed processor circuit 160 for analysis and control modification. Sensors 170 obtain gas sample measurements at a period frequency which reflects a possible period over which there may be significant changes, in order to ensure optimal performance of the unit 100 during peak CO2 saturation, and to save energy.

As of this writing, three popular methods for detecting CO2 include non-dispersive infrared sensors (NDIR), electromechanical sensors, and metal oxide semiconductor sensors. While any of these or other methods can be used together with the disclosure, NDIR is a particularly cost effective and efficient solution for incorporation into units 100. An NDIR is a spectroscopic sensor having principal components including an infrared source, a sample chamber, a light filter, and an infrared detector. Infrared light passes through the sampling chamber towards the detector. Likewise, there is another chamber with an enclosed reference gas, in this example CO2. Gas in the sample chamber causes absorption of light primarily at specific wavelengths, and the reduction of light at these wavelengths can be measured to determine the concentration of CO2. An optical filter can be used to eliminate light from wavelengths that are not of interest. In this manner, an amount of light detected indicates an extent of presence of CO2 molecules. An NDIR sensor can be used to detect other deleterious gases in accordance with the disclosure.

Gas filter 190 can be provided as an easily replaceable unit so that improved units can be provided over time as the relevant technology evolves. As of this writing, there are two principal filter technologies that can be used with the disclosure, and both can sequester carbon dioxide from the atmosphere. In particular the technologies include (1) filtration physically through the use of membranes or solid sorbents like zeolites or porous carbons, or (2) chemically through filtering with liquid amine, a derivative of ammonia, and (3) the use of metal-organic frameworks, which is a nascent technology as of this writing, but which can also be used with the disclosure, particularly as cost is reduced and efficiency is increased. Other filtration methods may arise which can be used to form gas filter 190.

Gas filter 190 can receive CO2 saturated air from intake fan 110, which air is passed through filter 190 at an optimal rate for efficient capture. For example, fan 110 should not drive air through filter 190 at a rate that is faster or slower than the CO2 can be captured at a given concentration. Air scrubbed of some or all of its contained CO2 is then expelled from unit 100 through an exhaust outlet 112 and into the surrounding atmosphere. When filter 190 is at or near capacity, it may be removed from unit 100 and brought to a facility for removal of the captured carbon. A fresh/empty filter 190 can be inserted when the loaded filter 190 is removed. Alternatively, when unit 100 is provided with the electric water heater 130, the filter can be partially or completely emptied of the captured carbon using a local temperature swing absorption method, for example, and the filtrate stored for later collection or appropriate disposal.

In one embodiment, unit 100 is assembled by assembling an intake fan 110, photovoltaic/solar cell 120, electric on-demand water heater 130, wireless communication circuit 140, processor circuit 160, sensors 170, including a gas sensor for the gas to be captured, energy storage/battery 180, and gas filter 190, into a frame 102. More particularly, processor circuit 160 and wireless communication circuit 14 are affixed to frame 102 and are connected to battery 180, or through a circuit connected to battery 180 which reduces volage to suitable level. A plurality of single speed fans are assembled onto a surface of frame 102, and connected to the battery 180 under control of the processor circuit 160 to operate individually and collectively. Solar cells 120 are mounted to an upper surface of frame 102 to gather sunlight and send electricity to battery 180 under control of processor circuit 160 which functions as a charge controller, or some other charge controller circuitry, as needed. Water heater 130 is affixed to frame 102 and connected a source of water and to battery 180 under control of processor 160. An output of water heater 130 is connected to filter 190 to pass hot water through filter 190 to enable purging. Gas sensor 190 is mounted to frame 102 to be exposed to the atmosphere without interference from other components of unit 100, and is electrically connected to processor circuit 160. Battery 180 is assembled into frame 102, and connected to components as described above, including having an output connected to any power conditioning circuity (not shown) as needed for the various power consuming components. Gas filter 190 is affixed to frame 102 to receive air from intake fan 110. Where purging of filter 190 is expected to be carried out away from unit 100, water heater 130 can be omitted. A source of electricity can be connected to either charge battery 180 or power unit 100. Battery 180 can be removed in this instance, although reliability could be adversely affected.

Example dimensions, in inches as width x height x depth, are as follows: unit 100 exterior dimensions, 36×42.5×48; intake fan 100, 10.5×10.5×5.5, gas filter 190, 12×34×34; battery 180 34×7×49; solar cell 120, 6×6; water heater 130, 8×12×4; wireless communication circuit 140, 10×10×3; sensors 170 as three housings, 8.5×4, 8.5×3.5, 4.5×3.5. It should be understood that these values represent a typical unit containing available components at low prices. A smaller size can typically be obtained by using more compact components, which tend to be more costly. Size is further dependent upon technology and availability. It is expected that sizes can be considerably smaller over time, particularly if there is widescale production of units 100. Further, while components are described as separate, electronics components can be combined onto a single circuit board, and mechanical components can be similarly constructed to fit closely together to form a compact module. In accordance with the disclosure, a wide variety of sizes can thus be used, however it is advantageous for unit 100 to be less than 200 pounds and fit upon a standard pallet (40×48×48), and ideally less than 100 pounds and less than half the size of a pallet (e.g. less than 20×48), and more favorably at less than 50 pounds and ¼^{th} the size of a pallet (e.g less than 20×24), although the disclosure can be carried out effectively with much larger sizes and greater weights, although the cost of deployment rises quickly with size and weight when it is contemplated to have numerous units 100.

The foregoing components are advantageously designed to be modular in nature, so that each may be removed or installed individually without specialized tools, or without tools at allow, to enable quick replacement as needed, and to facilitate future technology advancements.

Units are advantageously installed in highly populated areas, as close as practicable to CO2 generating sources. These sources can include homes, commercial facilities, vehicles, animals, and activities of people. These sources are generally low to the ground and near the general population. Accordingly, units 100 would be assembled into a hive or population where units 100 are closest to point sources, but not closer to each other than is necessary in order to carry out gas capture. In an embodiment, units 100 are designed to be installed alongside a typical air-conditioning unit or heating unit on the exterior of a home, apartment complex, or business, and can be sized and dimensioned to fit upon typical free space upon a concrete pad or mounting frame of an existing HVAC unit.

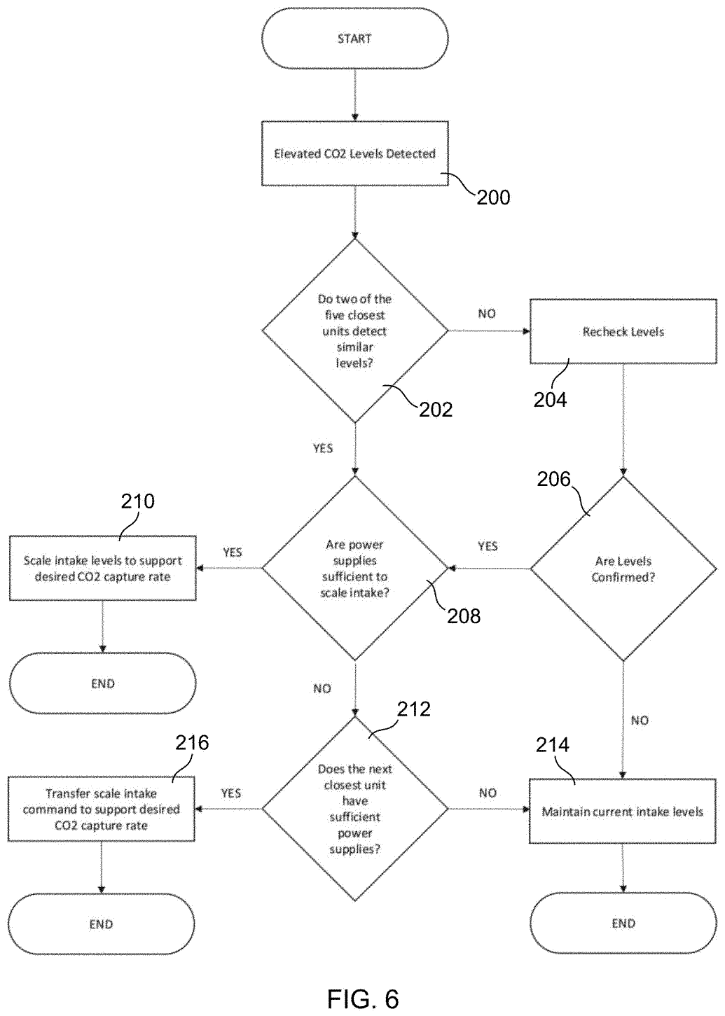

In an embodiment, two processes enable system 10 of the disclosure to operate efficiently as a networked and a closed system: a CO2 Detection, Scale & Intake Sharing Process, and a Unit Capacity Status and Intake Sharing Process. Process flow diagrams are provided for these processes in FIGS. 6-8.

The CO2 Detection, Scale & Intake Sharing Process detects atmospheric levels of CO2 (or other deleterious gas) using a sensor 190 that is a gas sensor of a first unit 100 (200), and validates against readings of two of five geographically nearest units 100 (202). If the levels are validated as being within a predetermined range, the first unit 100 can check if levels remain as initially sensed (204), and if so (206), and unit 100 is operationally ready (208), can either scale up air intake levels to capture more CO2 saturated air if the level is above a predetermined threshold (210), or reduce air intake to conserve energy for later use if the level is below a predetermined threshold.

If the first unit 100 is not operationally ready, The Unit Capacity Status and Intake Sharing Process will first evaluate the health and status of various components of first unit 100. If any of those components are out of tolerance or capacity, it will shift its intake responsibilities to the next closest and available unit, and issue a request for service through the network of units 100 and servers as described herein, while first unit 100 can continue as best as possible (214) until service can be carried out. Next, the process determines if one or more neighboring units 100 has an operational status (212), and if so, instructions are issued to the one or more neighboring units to increase capacity in consideration of a reduced capacity of the first unit 100 (216)s. The analysis of a status of first unit 100 and neighboring units can be carried out by processing circuit 160 of any interconnected unit 100, or by a server connected to the network of units 100 described elsewhere herein.

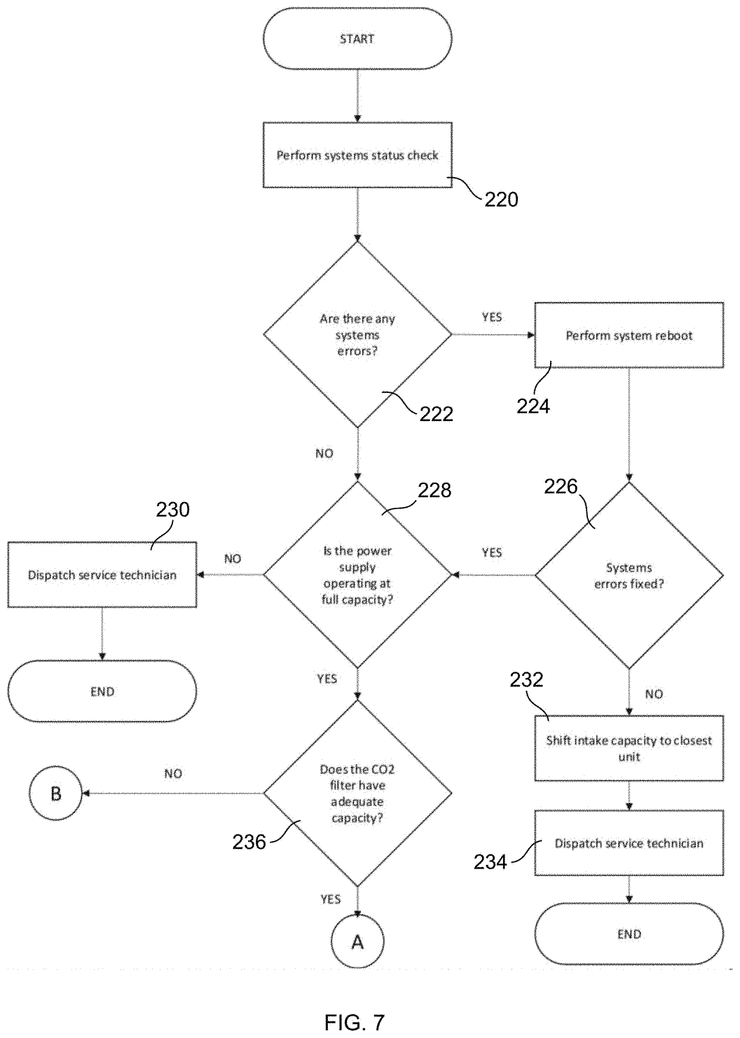

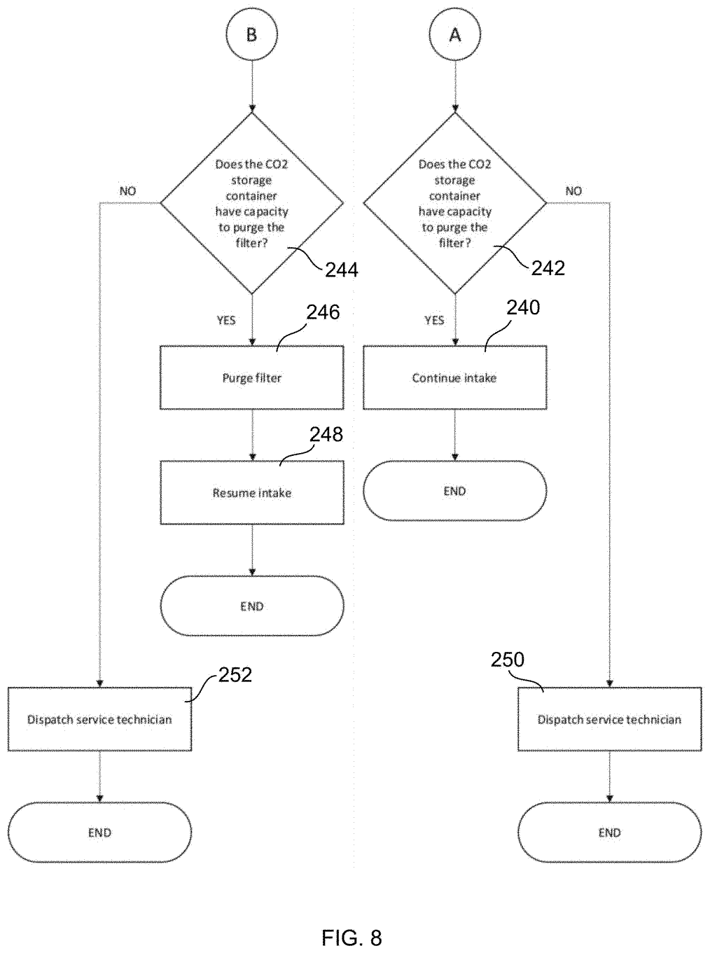

FIG. 7 illustrates a process for self-diagnostics of a unit 100. In particular, processor circuit 160 is connected to sensors 190 which can include voltage sensors, motion sensors, position sensors, and other sensors which can report information to processor circuit 160 of the state of the various components of unit 100. In addition, various components within unit 100 may include processors which can be connected to processor circuit 160 through wires or short range wireless communication, to report diagnostic or maintenance information. A system check (220) is therefore performed whereby processing circuit 160 gathers status information from other components within unit 100 as described herein. If there are any malfunctions or system errors (222), processor 160 can reboot (224) some or all electronic components within unit 100, and recheck (226). If rebooting does not success, intake capacity can be shifted to one or more geographically nearest units 100 (232), and if this is not successful, a technician can be dispatched (234) to troubleshoot the first unit, and the one or more nearest units. If there are no further errors, the power supply can be tested for capability (228), and if this test fails, a technician can be dispatched (230). If there were no errors, a capacity of the gas filter 190 can be tested (236). If it is working, the process can continue at FIG. 8 branch “A”, and continue intake (242). If it is desired to know in advance if purging will be capable, it may be checked in advance (242), and a technician dispatched if not (250). If gas filter 190 is at capacity, the process can continue at FIG. 8 branch “B”, where it is determined if there is an ability to purge filter 190 (244), and if there is, it is purged (246), and intake is resumed (248). If purging is not possible, a technician is dispatched (252).

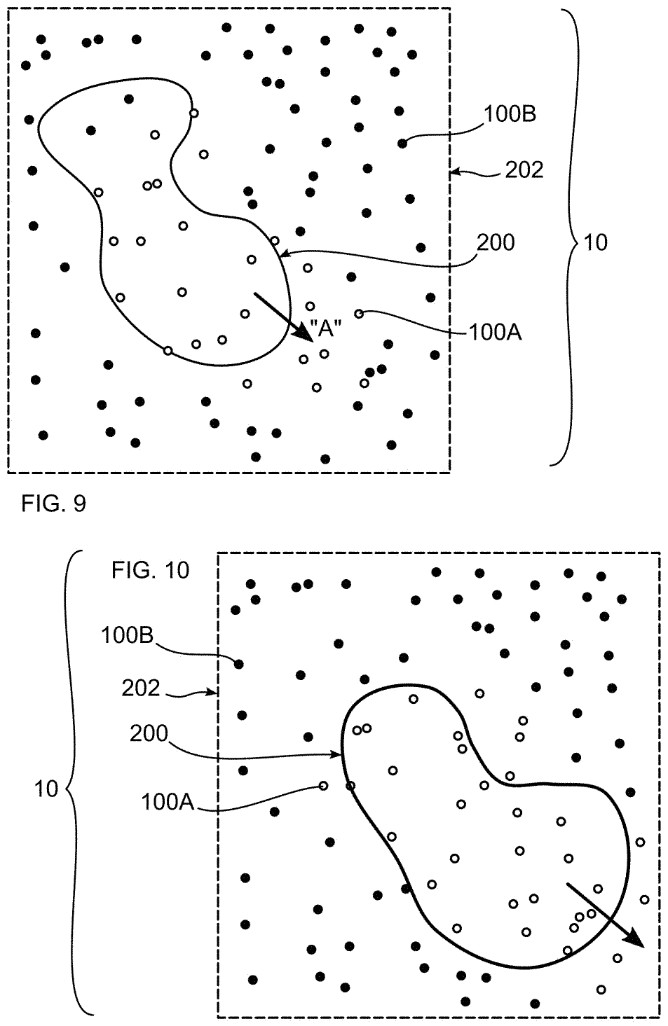

In FIG. 9, a dotted rectangle 202 indicates a portion of a geographic region in which system 10 is established, containing a plurality of units 100. Units 100 are diagrammatically represented as hollow circles corresponding to units 100A actively capturing gas, and filled-in circles corresponding to units 100B which are not actively capturing gas. Contour line 200 represents an area of higher deleterious gas concentration within the area. Arrow “A” indicates the general direction that contour line 200 is expected to move. As can be seen, more active units 100A lie inside contour 200 than outside. In FIG. 10, it may be seen that contour line 200 has moved as expected, and there continues to be more active units 100A within contour 200, as before, whereby units 100 are efficiently operating in areas of higher concentration.

A geographic region is advantageously as large as a city, a town, or at least a plurality of square miles within which the deleterious gas is being released in high concentrations. In such an area, units 100 can be distributed closer to known point sources, or distributed evenly if gas is released more or less evenly throughout the region. Distribution of units 100 can be affected by convenience places in which to position the units, for example existing HVAC pads or areas, or upon flat portions of roofs, or upon new pads, or upon scaffolding. Units 100 can further be positioned above existing a/c condensing units, whereby the air intake of the condensing unit draws more air into the vicinity of unit 100. Density of units 100 is dependent in part upon funds available for their purchase and deployment, and in some cases funds available for location rental. An advantageous density is at least one unit 100 per acre, although less than one unit per acre remains potentially effective, and more than one unit 100 can likewise be effective, particularly in dense urban areas, in which a plurality of units 100 per acre are advantageous. More particularly, in accordance with the disclosure, in a ‘swarm’ mode of operation, units 100 respond to behavior of their neighbor units 100, as well as ambient conditions, when determining an extent of their individual activity. Multiple units 100 form a colony which (a) responds to internal and external disturbances in an effort to capture deleterious gas, (b) work independently without centralized control, and (c) are self organized. In this manner, additional units 100 can be introduced without introduction to a central server, the newly added units 100 forming an efficient member of the colony on their own. A colony is formed from all units 100 which are in mutual communication over a common network. If the network is defined to be a lower power short range network, then the colony is defined as all units which are part of an unbroken chain of units 100 in mutual communication using the short range network. Such a colony can be expanded if at least one unit within the short range colony is in communication with another colony using a long range communication method, thereby potentially forming a new combined colony of cumulative size.

Each unit 100 is aware of the local concentration of deleterious gas due to sensors 170 including a gas sensor for the deleterious gas. It should be understood that processor 160 does not need to know the actual concentration of the gas based on sensor input, but only whether the concentration is high or low, or whether it is very high or low, medium high or low, somewhat high or low, etc. Units 100 also know how they are performing individually, by weighing or measuring captured gas within unit 100 over time.

Units 100 can further each know what operating parameters produced the highest yield of captured gas by comparing yield with operating parameters at one point of time with a given concentration and environmental conditions, and yield with differing operating parameters at another point of time with similar concentration and environmental conditions.

Operating parameters can include operating conditions under control of unit 100, including at least air intake rate, filter fullness, time of operation, and duration of operation. Other operating parameters include a functional state of the various components of unit 100, including whether components are non-functioning or marginally functioning.

Environmental conditions can include conditions not under control of unit 100, such as weather, including wind speed, precipitation, ambient temperature, and barometric pressure; as well as biogenic emissions, time of day, season, an extent of units within the colony performing at a reduced level, and environmental conditions experienced by other colony members.

One criteria for defining neighboring units includes those units 100 with which a given unit 100 can communicate using short range wireless communications. If this subset is large, it can be further reduced by a given number of units which have the highest wireless communication signal strength. Another criteria can be units whose location are known or determined to be close in some other manner, such as sensed GPS coordinates or locations provided. However, using signal strength alone, for example, furthers the autonomous operation of the colony. As units 100 share predefined data sets using wireless or other communication, a unit 100 can compare yield under given operating and environmental conditions with that of neighbors at the same point in time as the unit, and over a period of time. In this manner, a unit can determine the impact of its own operating parameters on those of a neighbor, and can optimize its own operating parameters in light of historical operating parameters and yields of neighbors, under various environmental parameters, to optimize yield individually and for the colony.

As such, each unit 100 can individually be active using operating parameters which are calculated to optimize yield both individually and collectively. Such calculations can be carried out with software algorithms using a straightforward approach, which can be simple or complex. Alternatively, such calculations can be made using artificial intelligence algorithms, which can produce the most flexible and efficient results over time.

In an embodiment, a maximum energy usage for a given yield can be determined, so that the colony always produces a net negative impact on the target gas. For example, for carbon capture, the amount of carbon captured by all operating units is compared with the carbon released to produce the energy needed to operate those units. Where the only energy used is produced by solar cells 120, the total carbon released is only the one-time carbon released to manufacture the units 100, as well as the ongoing carbon releases required to maintain the units, which includes emptying and gathering the captured gas from all units. Units 100 can be provided with sufficient reliability and purge storage 192 so that it can be known that a meaningful net negative result is obtained after maintenance is considered. Where there is energy input from utility sources, an amount of total energy being used, and thus carbon released, can be calculated for the colony. If the carbon released approaches within a predetermined limit to the carbon captured, units 100 producing lesser relative yield can be deactivated, until carbon release is below the limit.

Contour 200 can shift geographically, for example, due to a change in location of carbon release as well as due to environmental factors such as wind, temperature change, and rotation of the earth. Based on observations of neighbors, units 100 can determine if they are in an area of impending rise in concentration, or reduction in concentration; as such, units 100 can either take proactive measures to optimize performance, or reduce activity, respectively. Measures to optimize performance can include, for example, purging gas filter 190, performing a self-test or diagnostic, activating air intake, optimizing battery charge rate, obtaining network data, performing calculations, or carrying out any other energy intensive activity. Reducing activity can include, for example, slowing down or stopping intake, slowing down or temporarily stopping network activity, or reducing processor 160 power usage.

Through intelligent positioning of units 100 in populated areas where CO2 or other unwanted environmental gas concentrations are at their highest, system 10 can measure and validate gas concentrations as measured by a plurality of units 100, and can determine a maximally efficient energy consumption plan by operating an optimal number of units 100 distributed with respect to gas concentrations, each unit 100 operating at an optimal rate. This can significantly lower the cost of capture and energy consumed, maximizing the true carbon negative solution.

For example, if a unit 100 is impaired, for example it has a full filter that cannot be purged, yet a higher concentration of unwanted environmental gas is detected most proximate the impaired unit, several alternatives can be considered. The first is to operate one unit 100 geographically close to the impaired unit at a maximum output. However, the net captured gas will be lower since the concentration at the geographically close unit is not as high as it is near the impaired unit. Another option is to operate more than one nearby unit at maximum output. However, while the net capture may be higher, a greater amount of energy will be used. If the units are operating entirely on solar, this might be considered acceptable. However, excess energy derived from solar could be returned to the grid, reducing the generation of unwanted gases from electricity generation from other sources, such as combustion. Lastly, until the impaired unit is serviced, it may be most efficient to operate a plurality of units nearest the impaired unit, each at a lower than maximum rate, whereby the total energy usage is the same for a given amount of gas captured. To operate a given unit 100 at a lower rate, in an embodiment fans 110 may be operated at a slower speed, or if there are multiple fans 110, a subset of fans 110 can be operated, or a combination of fewer fans can be operated each at a slower speed. Software controlling the collective hive or swarm of units 100 can determine the optimal approach for the greatest gas capture at the lowest use of energy, in all scenarios. Artificial intelligence can be used to determine an optimal usage of a plurality of units 100 in each scenario.

By leveraging swarm intelligence (SI), system 10 leverages smaller more energy efficient units 100 (for example, relative to larger units positioned proximate point sources), that communicate with surrounding units 100. When elevated CO2 (or other unwanted environmental gas) levels are detected and then validated by nearby units 100, the entire system 10 engages as a collective to target CO2 concentrations for optimal capture. Likewise, when the collective detects reduced CO2 levels the system will reduce its total energy consumption and conserve energy for later use. Two additional energy usage scenarios follow.

Scenario 1—Individual units 100 are interconnected in a manner that allows them to optimize energy for the purpose of collecting atmospheric CO2 and/or other unwanted environmental gases. System 10 can determine it is more efficient to operate less than all fans 110 of a given unit, or to run one or more fans of a unit 100 at a lower speed, for example when concentrations near the unit 100 are low, or where a given unit 100 is found to be more efficient operating its fans at a less than maximal operating rate. Conversely, if it is determined that CO2 to energy consumption ratio is better by operating a plurality of fans in a given unit, or operating fans at a higher speed (rate), system 10 can pursue that option. This decision is determined by the collective and is based upon the measured CO2 concentration levels of each unit independently. Since CO2 levels may vary drastically from one unit 100 to the next, even when the units 100 are geographically nearby, the measured CO2 level at each unit is given proportional weight a decision regarding operation of all units 100 near a concentration.

Scenario 2—In the event a single unit 100 or small group of relatively nearby units 100 detect CO2 or other unwanted environmental gases at a level that is extreme/unusually high, all such units can be operated at a maximal rate, whereby all fans of a unit 100 are operated at a maximal speed, and if possible purging takes place at a maximal speed when required, until detecting of unwanted gases returns to normal or more typical levels for the area. System 10, operating as a collective, can additionally activate successive nearby units 100, even where concentrations may be lower or normal, to reduce demand on those units operating at a maximum level in the area of unusual concentration, thereby modifying adjacent concentrations in the area of unusual concentration, and additionally balancing the workload of the collective. Thus, where an extreme detected concentration measurement exceed an ability of a given unit to capture CO2 optimally, the collective of all units 100 work together to change the airflow patterns in a wider area, allowing the collective to bring the energy to capture ratio back into balance at an efficient overall rate.

By being part of a collective system 10, each unit 100 can be either (a) aware of the health/operating status of closest units to adapt its operating rate to maximize overall efficiency, or (b) controlled by a central server to adapt an operating rate of the given unit to maximize overall efficiency, depending on a mode of operation designated for the given unit. For example, if CO2 concentration levels are above a predefined energy efficient range, but the health of an affected given unit 100 is low, for example battery power is fully consumed, then neighboring units 100 can increase operating rate to compensate until the given unit captures sufficient energy via solar or is otherwise supplied with power. In an embodiment, such modifications to operation can take place independently by individual units 100, and can be monitored by a centralized system as a cross-check, and to enable study or artificial intelligence adaptation so that efficiency can be increased over time.

All references cited herein are expressly incorporated by reference in their entirety. There are many different features of the present disclosure and it is contemplated that these features may be used together or separately. Unless mention was made above to the contrary, it should be noted that all of the accompanying drawings are not to scale. Thus, the disclosure should not be limited to any particular combination of features or to a particular application of the disclosure. Further, it should be understood that variations and modifications within scope of the disclosure might occur to those skilled in the art to which the disclosure pertains. Accordingly, all expedient modifications readily attainable by one versed in the art from the disclosure set forth herein that are within the scope of the present disclosure are to be included as further embodiments of the present disclosure.

Reference Numbers:

10 system of units
100 gas capture unit
100A unit actively capturing
100B unit not actively capturing
102 unit frame
110 intake fan
112 exhaust outlet
120 solar cell
122 solar panel
130 water heater
140 wireless comm circuit
160 processor circuit
170 sensors
180 battery
190 gas filter
192 purge storage container
200 higher concentration contour
202 geographic region

What is claimed is:

1. A method for capturing a deleterious gas from atmospheric air, comprising:
positioning a plurality of deleterious gas capturing units within a region producing the deleterious gas, each unit including:
a filter of a type capable of capturing the deleterious gas from air when air is passed through the filter,
one or more fans for passing air from the atmosphere through the filter, the unit being in an active status when air is passed through the filter, and an inactive status when air is not being passed through the filter,
a sensor for sensing a level of the deleterious gas in the air,
a processor circuit connected to the sensor and the fans to obtain data from the sensor and control operation of the fan, and
a communication circuit responsive to the processor circuit to communicate data between the unit and at least one other unit; and
programming the processor of each unit to cause the communication circuit to communicate with a plurality of other units to communicate data including a concentration of deleterious gas in the air as measured by the sensor of the unit and whether the unit is in an active status;
programming the processor of each unit to independently determine an amount of air to pass through the filter based upon (a) a concentration of deleterious gas sensed by the sensor of the unit, and (b) a concentration of deleterious gas sensed by, as well as an activity status of, a plurality of other units in communication with the unit;
the independent determination based upon a calculation of maximizing yield of the unit and the plurality of units with which the unit is communicating.

2. The method of claim 1, wherein communicating with a plurality of other units includes communicating information pertaining to yield of the deleterious gas, and determining is further based upon yield of other units.

3. The method of claim 1, whereby the one or more fans are configured to have an adjustable rate of operation, and whereby independently determining an amount of air to pass through the filter includes determining a rate of operation of the one or more fans.

4. The method of claim 3, whereby determining a rate of operation of the one or more fans includes determining a number of fans which are operating.

5. The method of claim 3, whereby determining a rate of operation of the one or more fans includes determining a speed at which one or more fans are operating.

6. The method of claim 1, each unit further including a purge storage container and a source of hot water connected to the filter, hot water admissible into the filter under the control of the processor to purge the filter of components of the deleterious gas that have been captured and to pass the purged components into the purge storage container.

7. The method of claim 1, wherein the activity status includes whether one or more units are not functioning.

8. The method of claim 1, wherein the activity status includes whether one or more units are functioning at a maximal rate.

9. The method of claim 1, wherein the deleterious gas includes a carbon compound.

10. A method for capturing a deleterious gas from atmospheric air, comprising:
positioning a plurality of deleterious gas capturing units within a region producing the deleterious gas, each unit including:
a filter of a type capable of capturing compounds from the deleterious gas from air when air is passed through the filter,
one or more fans for passing air from the atmosphere through the filter, the unit being in an active status when air is passed through the filter, and an inactive status when air is not being passed through the filter,
a sensor for sensing a level of the deleterious gas in the air, and
a communication circuit; and
programming one or more electronic processors to:
a) communicate with each of a plurality of units to collect data from the sensor of each of the plurality of units using the communication circuit of each unit;
b) use the collected data to determine if the deleterious gas is present at a level above a predetermined level at each of the units;
c) determine at least one set of units of a predetermined set size, each of the units of the set having the predetermined level of deleterious gas present; and
d) communicate to each of the set of units to operate one or more fans of the unit to capture deleterious gas using the filter of the unit; and
e) periodically repeat steps (a)-(d) whereby units are operated only where and while the predetermined level of deleterious gas is present at a plurality of units.

11. The method of claim 10, each unit further including a battery configured to operate the one or more fans and sensor.

12. The method of claim 11, the one or more electronic processors further programmed to communicate with each of the plurality of units to determine an amount of battery capacity of the units, and using the determined battery capacity to determine members of the set of units of a predetermined size, the members of the set including only units having a predetermined amount of battery capacity.

13. The method of claim 10, wherein in step (d), the one more electronic processors communicate to each of the set of units a number of the one or more fans to be operated, or a rate of speed of the one or more fans to be operated, to achieve a total fan output corresponding to the predetermined level of deleterious gas present.

14. The method of claim 13, each unit further including a battery configured to operate the one or more fans and sensor, the number of fans to be operated or the rate of speed of the one or more fans corresponding to a charge level of the battery.

15. The method of claim 14, wherein if a first unit in the set of units has a charge level below a predetermined charge level, and a charge level of a unit not in the set of units that is near to the first unit is above a predetermined charge level, the one or more processors includes the unit that is near in the set.

16. The method of claim 10, each unit further including a means of removing captured gas compounds from the filter.

17. The method of claim 10, each unit further including a means of recording errors in the operation of the unit.

18. The method of claim 10, further including using the communication circuit to communicate the recorded errors to the one or more electronic processors.

19. The method of claim 18, the one or more electronic processors changing members of the set in response to recorded errors communicated.

20. The method of claim 10, wherein the one or more electronic processors changes the set of units based upon a predicted direction of movement of the predetermined level of deleterious gas away from the set based upon communication of deleterious gas levels communicated from units outside of the set.
